(19) United States
(12) Patent Application Publication
(10) Pub. No.: US 2010/0218776 A1
(43) Pub. Date: Sep. 2, 2010

(54) UPPER LIMB RESTRAINT DEVICE

(75) Inventors: Kazumasa Orihashi, Hiroshima-shi (JP); Shuro Hayashi, Hiroshima-shi (JP)

Correspondence Address: ROBERTS MLOTKOWSKI SAFRAN & COLE, P.C.;Intellectual Property Department, P.O. Box 10064, MCLEAN, VA, 22102-8064, US

(73) Assignee: JMS CO., LTD., Hiroshima-shi, Hiroshima (JP)

(21) Appl. No.: 12/161,332

(22) PCT Filed: Jan. 18, 2007
(86) PCT No.: PCT/JP2007/050705

§ 371 Date: Jul. 17, 2008

(30) Foreign Application Priority Data
Jan. 18, 2006 (JP) 2006-010070

(51) Int. Cl. A61F 5/37

(52) U.S. Cl. 128/879

(57) ABSTRACT

An upper limb restraint device 1 comprises a base 2 placed on the top surface of an upper limb rest 51, an upper limb rest engagement part 3 provided at an end of the base 2 close to the wrist joint 104 of an upper limb 100, an elbow joint supporting part 4 provided at the other end of the base 2 close to the elbow joint 102, and two strings 5 and 6. The upper limb rest engagement part 3 is formed in the shape of a pocket capable of receiving an end of the upper limb rest 51. The elbow joint supporting part 4 is formed to cover the elbow joint 102 of the upper limb 100 laid on the upper limb rest 51. The root ends of the strings 5 and 6 are attached to the elbow joint supporting part 4. The strings 5 and 6 are bound around the upper limb rest 51 with the elbow joint supporting part 4 covering the elbow joint 102.

### TECHNICAL FIELD

This invention relates to upper limb restraint devices for use in restraining upper limbs of patients under surgery on upper limb rests.

### BACKGROUND ART

Generally, patients undergoing surgery, such as on their chests or abdomens, are strapped under anesthesia to surgical beds to avoid their sudden movement. In strapping such a patient to a surgical bed, depending on the type of surgery, one or both of the upper limbs of the patient may be restrained on an upper limb rest or rests attached to the surgical bed after one or both of the upper limbs are taken away from his (or her) body, then raised out from his side or sides and then further raised up to one or both sides of his face by bending his elbow joint or joints, i.e., after the patient is brought into a so-called “arm-raised” position. The top surface of the upper limb rest is located slightly above the patient's face lying supine on the surgical bed and inclines so that the elbow joint of his upper limb is below the wrist joint thereof.

In the case of restraining a patient's upper limb on an upper limb rest with the upper limb raised up, such an upper limb restraint device as disclosed in Patent Document 1 may be used. The upper limb restraint device includes a fabric body formed in the shape of a strip and engagement parts provided at both longitudinal ends of the body and composed, such as of hook and button fastening. The body width is selected at a value wide enough to cover the upper limb from its wrist joint to its elbow joint. In using the upper limb restraint device, the upper limb is laid on the top surface of the upper limb rest, the body of the upper limb restraint device is then wrapped around the upper limb and the upper limb rest together and the engagement parts are then engaged.

Patent Document 1: Published Japanese Patent Application No. H06-319749

### DISCLOSURE OF THE INVENTION

### Problems to be Solved by the Invention

After patients are given anesthesia, their blood pressures need to be constantly monitored by placing blood pressure monitoring cannulas in radial arteries near the wrist joints of their upper limbs. With the use of such a wide upper limb restraint device as in Patent Document 1 for the patient, however, the position of placement of the blood pressure monitoring cannula may not be viewed since his upper limb is covered to the vicinity of the wrist joint with the upper limb restraint device. If in this state the blood pressure monitoring cannula is pulled out of the radial artery, the patient bleeds invisibly to the anesthetist, which may delay blood stanching.

Furthermore, during surgery, an intravenous line in the upper limb often needs to be established for fluid replacement through his veins. Since, however, the upper limb restraint device disclosed in Patent Document 1 is wide as stated above, it interferes with the establishment of an intravenous line for fluid replacement. Therefore, in order to establish an intravenous line for fluid replacement, the above upper limb restraint device must be detached and fitted again, which is a troublesome work.

Furthermore, when a patient's upper limb is restrained on the upper limb rest with the upper limb raised up, the upper limb is more likely to slide down from the upper limb rest, such as because of his weight or body movement, than when restrained with the upper limb placed on the side of his body. If the upper limb slides down from the upper limb rest, the blood pressure monitoring cannula or the intravenous line for fluid replacement might be pulled out, which affects the surgical procedure. Therefore, in order to prevent the upper limb from sliding off, the above upper limb restraint device needs to be securely wrapped around the rest while compressing the upper limb. When the upper limb is compressed too much, however, this may incur blood circulation disorder or nerve disorder and may disable blood pressure monitoring via the blood pressure monitoring cannula and fluid replacement via the newly established intravenous line. Since, thus, it is difficult to adjust the strength of wrapping of the upper limb restraint device of Patent Document 1 around the upper limb, there arise a problem that it takes a long time to restrain the upper limb and a problem that, for example, a person of little experience cannot restrain the upper limb well.

The present invention has been made in view of the foregoing points and, therefore, an object of the present invention is that in restraining a patient's upper limb on an upper limb rest during surgery, even a person of little experience can restrain the upper limb on the upper limb rest simply and in a short time, with a wide region of the upper limb around the wrist joint exposed and without tightly compressing the upper limb, to prevent the upper limb from sliding off from the upper limb rest.

### Means to Solve the Problems

To attain the above object, in the present invention, a base placed between the upper limb rest and the upper limb is provided with an upper limb rest engagement part engageable with the upper limb rest and an elbow joint supporting part for covering the elbow joint and a string extending from the elbow joint supporting part is bound around the upper limb rest, whereby the upper limb can be restrained on the upper limb rest.

Specifically, a first aspect of the invention is directed to an upper limb restraint device for restraining an upper limb of a patient under surgery on an upper limb rest with the upper limb raised. The upper limb restraint device includes: a base placed between the upper limb rest and the upper limb; an upper limb rest engagement part provided at an end of the base close to the wrist joint of the upper limb and configured to engage with an end of the upper limb rest close to the wrist joint; an elbow joint supporting part provided at the other end of the base close to the elbow joint of the upper limb and configured to cover the elbow joint of the upper limb laid on the upper limb rest from the underside to the upper side of the elbow joint; and a string extending from a portion of the elbow joint supporting part covering the upper side of the elbow joint and capable of being bound around the upper limb rest.

With the above configuration, the base is held to the upper limb rest by engaging the upper limb rest engagement part with the upper limb rest. Then, the elbow joint of the upper limb is covered by the elbow joint supporting part of the base held to the upper limb rest and the string is then bound around the upper limb rest, whereby the elbow joint of the upper limb is held against the upper limb rest from the upper side of the elbow joint supporting part and certainly supported by the elbow joint supporting part. Thus, the upper limb can be prevented, without being tightly compressed, from sliding off from the upper limb rest. In restraining the upper limb on the upper limb rest, it is sufficient if the elbow joint is covered by the elbow joint supporting part and the string is then bound around the upper limb rest. Therefore, the work of restraining the upper limb can be carried out simply and in a short time. Furthermore, since the upper limb is restrained on the upper limb rest by supporting the elbow joint, the upper limb can be exposed over a wide region thereof around the wrist joint.

A second aspect of the invention is the upper limb restraint device according to the first aspect of the invention, wherein the upper limb rest engagement part is formed in the shape of a pocket capable of covering a portion of the upper limb rest close to the wrist joint.

With the above configuration, the upper limb rest engagement part can be easily engaged with the upper limb rest.

A third aspect of the invention is the upper limb restraint device according to the first aspect of the invention, wherein the base is made of fabric and the elbow joint supporting part is formed by folding the fabric.

With the above configuration, the elbow joint supporting part can be easily formed.

A fourth aspect of the invention is the upper limb restraint device according to the first aspect of the invention, wherein the string comprises a plurality of said strings extending from separate points on the elbow joint supporting part.

With the above configuration, the elbow joint of the upper limb restrained on the upper limb rest is stabilized.

A fifth aspect of the invention is the upper limb restraint device according to the first aspect of the invention and further including a warming device that warms the upper limb.

With the above configuration, the upper limb restrained on the upper limb rest is warmed, which enables to restrain the body temperature of the patient during surgery from dropping.

A sixth aspect of the invention is the upper limb restraint device according to the fifth aspect of the invention, wherein the warming device is formed by containing a gel warming material capable of warming the upper limb in a bag member and disposed between the upper limb rest and the upper limb.

With the above configuration, the warming material can restrain the body temperature of the patient during surgery from dropping. Furthermore, since the warming material flows through the interior of the bag member to give cushioning, it can be avoided that the upper limb locally forcibly bears against the upper limb rest.

A seventh aspect of the invention is the upper limb restraint device according to any one of the first to sixth aspects of the invention and further including a cushion member placed between the upper limb rest and the upper limb.

With the above configuration, it can be avoided that the upper limb locally forcibly bears against the upper limb rest.

An eighth aspect of the invention is the upper limb restraint device according to the seventh aspect of the invention, wherein the cushion member comprises a bag member into which air is injected.

With the above configuration, the cushion member has a light weight and a simple structure.

A ninth aspect of the invention is the upper limb restraint device according to the seventh aspect of the invention, wherein the cushion member is restrained together with the upper limb on the upper limb rest by the string.

With the above configuration, a special member for fixing the cushion member to the upper limb rest is dispensed with.

A tenth aspect of the invention is the upper limb restraint device according to the first aspect of the invention, wherein the base includes a hollow part.

With the above configuration, since the hollow part is disposed between the upper limb rest and the upper limb, it can be avoided that the upper limb locally forcibly bears against the upper limb rest.

An eleventh aspect of the invention is the upper limb restraint device according to the tenth aspect of the invention, wherein the hollow part is provided with an inflation valve through which air is injected into the hollow part.

With the above configuration, the upper limb restraint device can be carried to the point of use in a compact form without injecting air into the hollow part before use and then inflated with air through the inflation valve at the point of use to form the hollow part.

A twelfth aspect of the invention is the upper limb restraint device according to the tenth aspect of the invention, wherein the hollow part comprises at least three said hollow parts arranged side by side in a width direction of the upper limb rest and the hollow parts at both ends of the base in the width direction of the upper limb rest are formed to have a larger size than the hollow part in the middle of the base.

With the above configuration, when the upper limb is laid on the upper limb rest after the upper limb restraint device is set on the upper limb rest, the upper limb sinks between the hollow parts at both ends of the base in the width direction of the upper limb rest.

### EFFECTS OF THE INVENTION

According to the first aspect of the invention, the upper limb rest engagement part can be engaged with the upper limb rest, the elbow joint supporting part can cover the elbow joint and the string can be bound around the upper limb rest from above the elbow joint. Therefore, the upper limb can be restrained on the upper limb rest, without being tightly compressed, to prevent from sliding off from the upper limb rest. Thus, blood circulation disorder and nerve disorder in the upper limb can be avoided and compression of the blood pressure monitoring cannula and the intravenous line for fluid replacement can be avoided, thereby providing normal operation of blood monitoring and fluid replacement. In addition, even a person of little experience can carry out the work of restraining the upper limb simply and in a short time. Furthermore, the upper limb restrained on the upper limb rest can be exposed over a wide region thereof around the wrist joint. Therefore, the blood pressure monitoring cannula can be viewed and, even if it is pulled out of the artery, this can be addressed promptly. In addition, a new intravenous line for fluid replacement can be established without detaching and fitting again the upper limb restraint device.

According to the second aspect of the invention, since the upper limb rest engagement part is formed in the shape of a pocket, the upper limb rest engagement part can be easily engaged with the upper limb rest, which further simplifies the work of restraining the upper limb.

According to the third aspect of the invention, the elbow joint supporting part can be readily obtained by simply folding the fabric making the base, which reduces the cost of the upper limb restraint device.

According to the fourth aspect of the invention, since the string comprises a plurality of strings, the elbow joint can be supported more stably by the elbow joint supporting part, which prevents the upper limb from sliding off from the upper limb rest.

According to the fifth aspect of the invention, since the body temperature of the patient during surgery can be restrained from dropping, the revitalization of the patient after surgery can be promoted.

According to the sixth aspect of the invention, since the warming device is formed by containing a gel warming material in a bag member, this reduces the invasion of the upper limb in restraining it on the upper limb rest.

According to the seventh aspect of the invention, since the cushion member can be placed between the upper limb rest and the upper limb, this reduces the invasion of the upper limb in restraining it on the upper limb rest.

According to the eighth aspect of the invention, since the cushion member comprises an air-injected bag member, it can have a light weight and a simple structure, which facilitates the handling of the upper limb restraint device.

According to the ninth aspect of the invention, the cushion member can be easily restrained on the upper limb rest.

According to the tenth aspect of the invention, since the hollow part can be disposed between the upper limb rest and the upper limb, this reduces the invasion of the upper limb in restraining it on the upper limb rest.

According to the eleventh aspect of the invention, the upper limb restraint device can be carried to the point of use in a compact form and then used.

According to the twelfth aspect of the invention, since the hollow parts at both ends of the base in the width direction of the upper limb rest have a larger size than the hollow part in the middle of the base, the upper limb restrained on the upper limb rest can be further stabilized.

### BRIEF DESCRIPTION OF DRAWINGS

FIG. 1 is a side view illustrating a state of use of an upper limb restraint device according to Embodiment 1 of the invention.

FIG. 2 is a plan view illustrating the state of use of the upper limb restraint device.

FIG. 3 is a view of the upper limb restraint device from an opening.

FIG. 4A is a plan view of a cushion member, FIG. 4B is a view of the cushion member from a longitudinal end thereof and FIG. 4C is a side view of the cushion member.

FIG. 5 is a side view showing a state that the cushion member and the upper limb restraint device are set on an upper limb rest.

FIG. 6 is a corresponding view of FIG. 5, showing a state that an upper limb is laid on the upper limb restraint device.

FIG. 7 is a corresponding view of FIG. 5, showing a state that the upper limb is restrained on the upper limb rest.

FIG. 8 is a corresponding view of FIG. 4, showing a modification of the cushion member.

FIG. 9 is a plan view of an upper limb restraint device according to Embodiment 2 of the invention.

FIG. 10 is a cross-sectional view taken along the line X-X of FIG. 9.

FIG. 11 is a cross-sectional view taken along the line XI-XI of FIG. 9.

FIG. 12 is an enlarged cross-sectional view of a film constituting part of the upper limb restraint device according to Embodiment 2.

FIG. 13 is a corresponding view of FIG. 5, showing the upper limb restraint device according to Embodiment 2.

FIG. 14 is a corresponding view of FIG. 7, showing the upper limb restraint device according to Embodiment 2.

FIG. 15 is a corresponding view of FIG. 9, showing an upper limb restraint device according to Modification 1 of Embodiment 2.

FIG. 16 is a corresponding view of FIG. 9, showing an upper limb restraint device according to Modification 2 of Embodiment 2.

### LIST OF REFERENCE NUMERALS

1 upper limb restraint device
2 base
3 upper limb rest engagement part
4 elbow joint supporting part
5 first string
6 second string
10 cushion member
30-32 hollow part
40 inflation valve
50 surgical bed
51 upper limb rest
100 upper limb
102 elbow joint
104 wrist joint

### BEST MODE FOR CARRYING OUT THE INVENTION

A detailed description is given below of embodiments of the present invention with reference to the drawings. The following description of the preferred embodiments is merely illustrative in nature and is not intended to limit the scope, applications and use of the invention.

### Embodiment 1

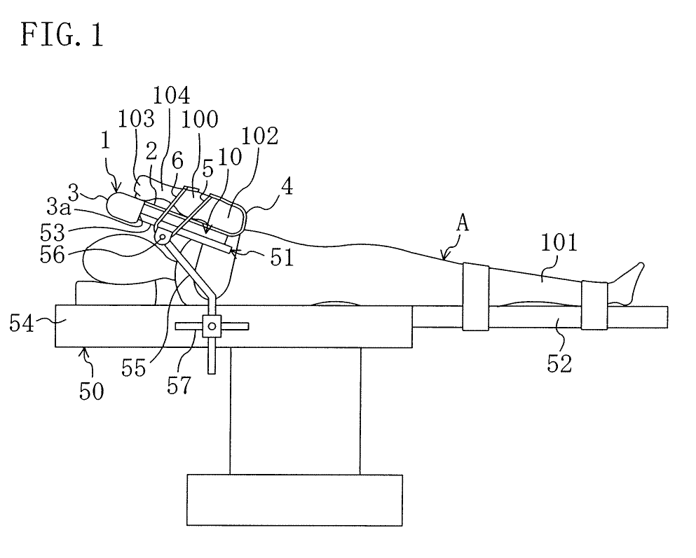

FIG. 1 shows a state of use of an upper limb restraint device 1 according to Embodiment 1 of the invention. In the description of Embodiment 1, the structure of a surgical bed 50 is described prior to the description of the structure of the upper limb restraint device 1. The surgical bed 50 includes upper limb rests 51 for restraining upper limbs 100 and a lower limb rest 52 for restraining lower limbs 101 and is the same as used in the general medical field.

Each upper limb rest 51 includes a hard plate-shaped member 53 and a support rod 55 supporting the plate-shaped member 53 to the body 54 of the surgical bed 50. The plate-shaped member 53 is used for the purpose of stabilizing the distal side of the upper limb 100 including the elbow joint 102 and is formed in a substantially rectangular shape slightly longer than the distance from the hand 103 to the elbow joint 102 of the upper limb 100. The width of the plate-shaped member 53 is selected at a wider value than the thickness of the upper limb 100.

The support rod 55 is formed to extend substantially vertically. The upper end of the support rod 55 is pivotally attached to a horizontally extending pivot shaft 56 mounted to substantially the longitudinal midportion of the under surface of the plate-shaped member 53. Thus, the plate-shaped member 53 can be fixed as it is pivotally moved to any desired position. On the other hand, a lower part of the support rod 55 is secured through a positioning mechanism 57 to the side of the body 54. The positioning mechanism 57 is configured to allow the support rod 55 to move in the longitudinal direction of the surgical bed 50, i.e., in the direction of body height of the patient A, and in the vertical direction and to fix the support rod 55 moved to any desired position. The lower limb rest 52 is configured to open to the right and left with respect to the patient A lying supine on the surgical bed 50.

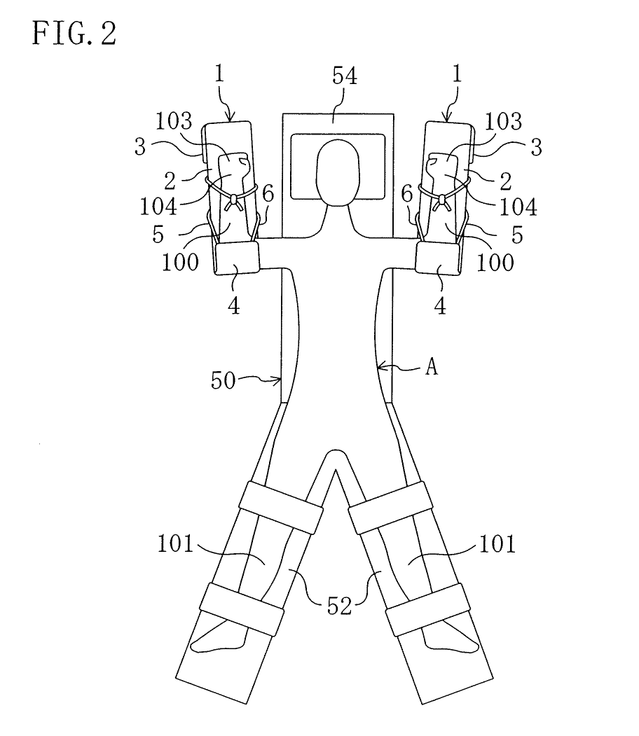

Furthermore, the plate-shaped members 53 are positioned, with their longitudinal direction conforming to the direction of body height of the patient A, just beside the face of the patient A lying supine on the surgical bed 50 in plan view as shown in FIG. 2 and slightly above the face in side view as shown in FIG. 1. The plate-shaped members 53 are fixed inclined to become lower with approach to the lower limbs 101 of the patient A.

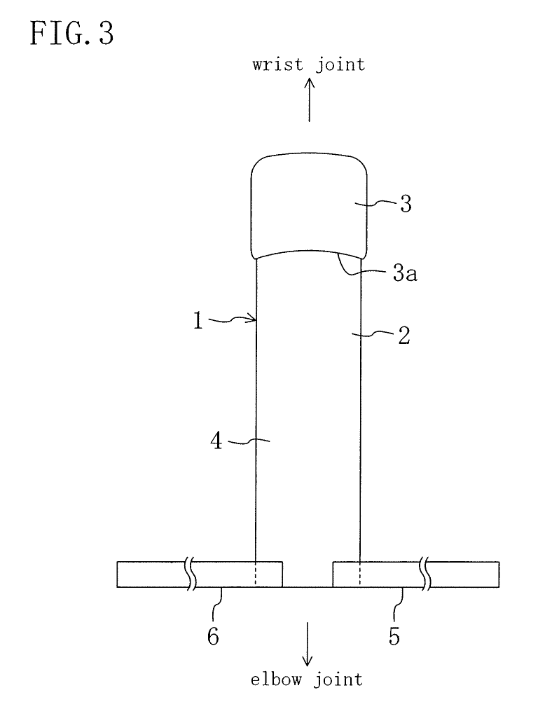

Next, a description is given of the structure of the upper limb restraint device 1. The upper limb restraint device 1, as also shown in FIG. 3, includes a base 2, an upper limb rest engagement part 3, an elbow joint supporting part 4 and first and second strings 5 and 6. The base 2, the upper limb rest engagement part 3 and the elbow joint supporting part 4 are made up of a sheet of fabric. Examples of the fabric include cotton fabric and chemical fiber fabric.

The base 2 is, as shown in FIGS. 1 and 2, placed between the top surface of the plate-shaped member 53 and the upper limb 100 and formed in a substantially rectangular shape to cover the entire top surface of the plate-shaped member 53. The upper limb rest engagement part 3 is provided at one end of the base 2 close to the wrist joint 104 of the upper limb 100. The upper limb rest engagement part 3 is obtained by folding an end of the fabric close to the wrist joint 104 back to the elbow joint 102 and seaming both lateral ends of the folded part to form a pocket having an opening 3a only at its end close to the elbow joint 102. The diameter of the opening 3a of the upper limb rest engagement part 3 is selected at a value at which the end of the plate-shaped member 53 close to the wrist joint 104 can be loosely inserted into it. The depth of the upper limb rest engagement part 3 is selected at such a value that the plate-shaped member 53 cannot be easily pulled out of it once the plate-shaped member 53 has been inserted in it.

The elbow joint supporting part 4 is continued to the other end of the base 2 close to the elbow joint 102 of the upper limb 100. The elbow joint supporting part 4 is formed to cover the elbow joint 102 of the upper limb 100 laid on the upper limb rest 51 from the underside to the upper side of the elbow joint 102. The size of the elbow joint supporting part 4 in the width direction of the plate-shaped member 53 is selected at substantially the same value as the size of the base 2 in the same direction.

The first and second strings 5 and 6 are made of fabric and extend from the end of the elbow joint supporting part 4 opposite to the base 2. The root end of the first string 5 is sewed on one lateral end of the elbow joint supporting part 4, while the second string 6 is sewed on the other lateral end of the elbow joint supporting part 4. The first and second strings 5 and 6 have substantially equal lengths. However, the first and second strings 5 and 6 may have different lengths. Since the first and second strings 5 and 6 are made of fabric, they can be cut with ease if they are too long.

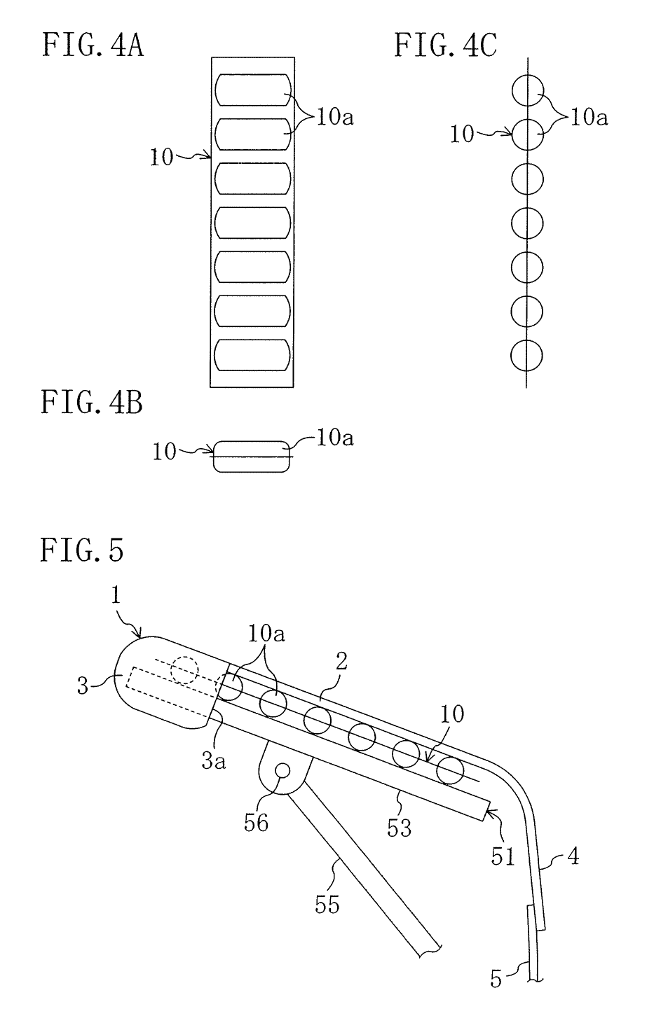

The upper limb restraint device 1 further includes a cushion member 10 placed between the upper limb rest 51 and the upper limb 100. The cushion member 10 comprises a bag member obtained by laminating two resin films together and is formed to extend from the vicinity of one longitudinal end to the vicinity of the other longitudinal end of the plate-shaped member 53. As shown in FIG. 4, the cushion member 10 has a plurality of cylindrical parts 10a formed at longitudinally spaced intervals to extend substantially transversely. Parts of the two films surrounding the cylindrical parts 10a are bonded to each other and each cylindrical part 10a is filled with air.

Next, a description is given of restraint of the upper limbs 100 on the upper limb rests 51 with the use of the upper limb restraint devices 1 having the above structure. First, as shown in FIG. 5, each upper limb restraint device 1 is set on the upper limb rest 51. Specifically, the cushion member 10 is put on the top surface of each plate-shaped member 53 and a part of the plate-shaped member 53 close to the wrist joint 104 is then inserted into the opening 3a of the upper limb rest engagement part 3 of the upper limb restraint device 1 to engage the upper limb rest engagement part 3 with the plate-shaped member 53, thereby holding the base 2 to the upper limb rest 51. In this case, since the upper limb rest engagement part 3 has the shape of a pocket, its engagement with the upper limb rest 51 can be easily achieved.

Furthermore, a part of the cushion member 10 close to the wrist joint 104 is inserted into the upper limb rest engagement part 3 from the opening 3a. Thus, the cushion member 10 is held to the upper limb rest 51 and held against its displacement. Then, the base 2 is put on the top surface of the cushion member 10. The elbow joint supporting part 4 and the first and second strings 5 and 6 are still left hanging from the end of the upper limb rest 51 close to the elbow joint 102.

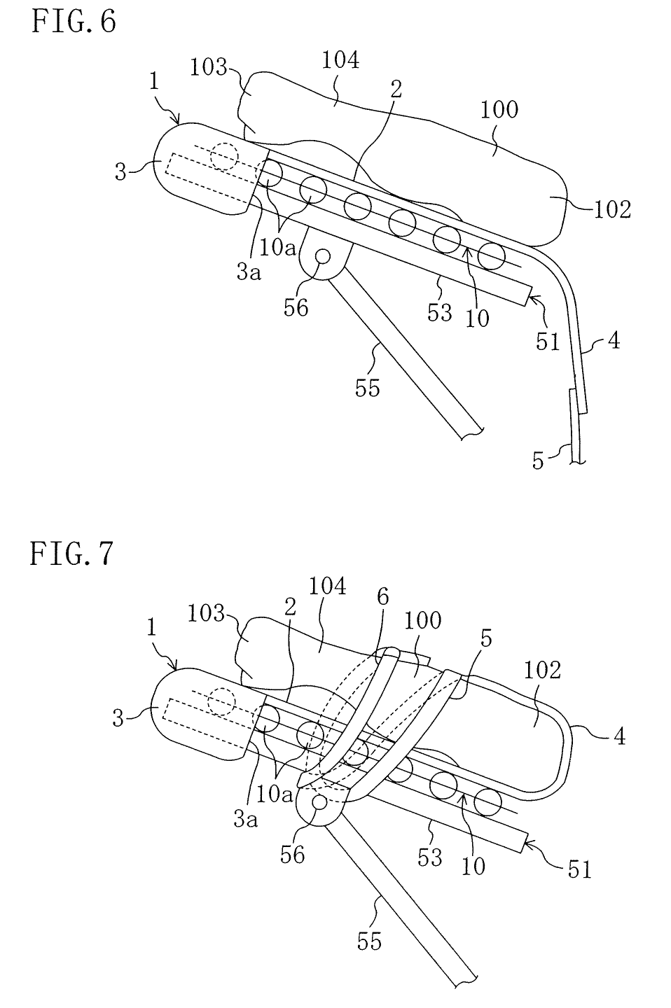

Thereafter, each upper limb 100 of the patient A lying supine on the surgical bed 50 is taken away from his body, then raised out from his side, then further raised up to the side of his face by bending the elbow joint 102 to bring the upper limb 100 into an arm-raised position, and then laid on the top surface of the base 2 of the upper limb restraint device 1 as shown in FIG. 6. In this case, since the upper limb rest 51 is inclined, the end of the upper limb 100 close to the elbow joint 102 is located below the other end thereof close to the wrist joint 104.

Next, as shown in FIG. 7, the elbow joint supporting part 4 of the upper limb restraint device 1 is folded upward to cover the elbow joint 102 of the upper limb 100 from its underside to upper side. Thereafter, the first string 5 is wound around the upper limb 100 to under the plate-shaped member 53, while the second string 6 is wound around the upper limb 100 to under the plate-shaped member 53 from the opposite side to the first string 5, i.e., from the side of the upper limb 100 close to the second string 6. These first and second strings 5 and 6 are crossed under the plate-shaped member 53 and entwined around the upper end of the support rod 55 at a point closer to the wrist joint 104 than to the elbow joint 102. Subsequently, the first string 5 is further wound towards the second string 6 and taken to the upper side of the upper limb 100, while the second string 6 is further wound towards the first string 5 and taken to the upper side of the upper limb 100. The first and second strings 5 and 6 thus wound around the upper limb rest 51 are tied together above the upper limb 100, thereby completing the work of restraining the upper limb 100 on the upper limb rest 51.

In this state that the upper limb 100 is restrained on the upper limb rest 51, the first and second strings 5 and 6 extend from above the elbow joint supporting part 4. Therefore, the strings 5 and 6 hold the elbow joint 102 against the upper limb rest 51 from the upper side of the elbow joint supporting part 4, whereby the elbow joint 102 can be certainly supported by the elbow joint supporting part 4. In addition, the upper limb 100, which would otherwise be liable to slide down because of the elbow joint 102 locating below the wrist joint 104, is supported by the elbow joint supporting part 4 to cover it from its underside with the elbow joint supporting part 4. Therefore, the elbow joint 102 is difficult to slip out of the elbow joint supporting part 4. As a result of these points, the upper limb 100 can be prevented, without being tightly compressed by the first and second strings 5 and 6, from sliding off from the upper limb rest 51. Furthermore, the cushion member 10 is restrained on the upper limb rest 51 together with the upper limb 100 by the first and second strings 5 and 6.

In restraining the upper limb 100 on the upper limb rest 51, it is sufficient if the elbow joint 102 is covered by the elbow joint supporting part 4 and the first and second strings 5 and 6 are then wound around the upper limb rest 51 and tied together. Therefore, the work of restraining the upper limb 100 can be carried out simply and in a short time. Furthermore, since the upper limb 100 is restrained on the upper limb rest 51 by supporting the elbow joint 102, the upper limb 100 can be exposed over a wide region thereof around the wrist joint 104.

As described so far, in the upper limb restraint device 1 according to this embodiment the upper limb rest engagement part 3 is engaged with the plate-shaped member 53 of the upper limb rest 51, the elbow joint 102 is covered by the elbow joint supporting part 4 and the first and second strings 5 and 6 are bound around the upper limb rest 51 from above the elbow joint 102. Therefore, the upper limb 100 can be restrained on the upper limb rest 51, without being tightly compressed, to prevent from sliding off from the upper limb rest 51. Thus, blood circulation disorder and nerve disorder in the upper limb 100 can be avoided and compression of the blood pressure monitoring cannula and the intravenous line for fluid replacement can be avoided, thereby providing normal operation of blood monitoring and fluid replacement. In addition, even a person of little experience can carry out the work of restraining the upper limb 100 simply and in a short time. Furthermore, the upper limb 100 restrained on the upper limb rest 51 can be exposed over a wide region thereof around the wrist joint 104. Therefore, the blood pressure monitoring cannula can be viewed and, even if it is pulled out of the artery, this can be addressed promptly. In addition, a new intravenous line for fluid replacement can be established without detaching and fitting again the upper limb restraint device 1.

Furthermore, since the upper limb rest engagement part 3 is formed in the shape of a pocket, the upper limb rest engagement part 3 can be easily engaged with the upper limb rest 51, which further simplifies the work of restraining the upper limb 100.

Furthermore, since the base 2 is made of fabric and the elbow joint supporting part 4 is formed by simply folding the fabric, the elbow joint supporting part 4 can be readily obtained, which reduces the cost of the upper limb restraint device 1.

Furthermore, since the first and second strings 5 and 6 are disposed away from each other on the elbow joint supporting part 4, the elbow joint 102 can be supported more stably by the elbow joint supporting part 4, which prevents the upper limb 100 from sliding off from the upper limb rest 51.

Furthermore, since the cushion member 10 is placed between the upper limb rest 51 and the upper limb 100, the upper limb 100 can be prevented from locally forcibly bearing against the plate-shaped member 53, which reduces the invasion of the upper limb 100 in restraining it on the upper limb rest 51.

Furthermore, since the cushion member 10 comprises an air-injected bag member, it can be formed to have a light weight and a simple structure, which facilitates the handling of the upper limb restraint device 1.

Furthermore, since the cushion member 10 is restrained on the upper limb rest 51 together with the upper limb 100 by the first and second strings 5 and 6, it can be easily fixed to the upper limb rest 51.

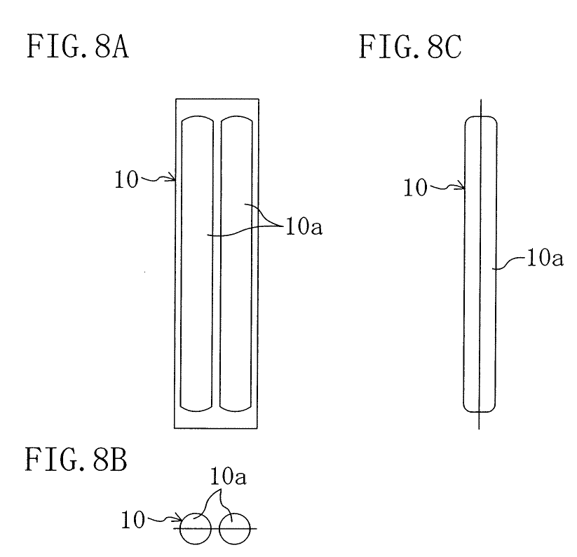

The cushion member 10 may be configured, as for example in a modification shown in FIG. 8, so that two cylindrical parts 10a are formed to extend long in the longitudinal direction of the cushion member 10 and arranged side by side in the transverse direction of the cushion member 10. The number of cylindrical parts 10a of the cushion member 10 and the configuration and size of each cylindrical part 10a can be appropriately selected. Furthermore, the cushion member 10 can be made, such as of resin material including urethane foam and polystyrene foam, synthetic rubber, natural rubber, thick fabric.

The cylindrical part 10a of the cushion member 10 may contain a gel warming material (warming device). An example of such a warming material used in this case is a material obtained by impregnating water-absorbing polymer made of a derivative of plant cellulose extracted such as from conifer with water. When impregnated with water, the plant cellulose derivative has a much higher viscosity than water and becomes gelatinous. When, thus, the warming material is a material in which a plant cellulose derivative is impregnated with water, its safety to the human body is high. Therefore, in case where the warming material leaks out, this does not have a significant affect on the human body. Furthermore, plant cellulose derivatives can be easily biodegraded by microorganisms existing such as in soil in nature and, therefore, can be easily disposed of. The warming device may be, for example, an electric heater using a heating wire or a ceramic heater.

When the upper limb 100 is warmed by the warming material, the blood flowing through the upper limb 100 is not cooled during surgery, thereby restraining the drop in body temperature. Thus, out of energies of the patient A, the energy used to increase the body temperature can be reduced to store a larger amount of energy used for revitalization after surgery. In addition, since the drop in body temperature is restrained, impairment of the immune system can be avoided to reduce the risk of the patient A being infected by a pathogenic organism. As a result of these points, the revitalization of the patient A after surgery can be promoted. Furthermore, since the flow of the warming material gives cushioning, this avoids that the upper limb 100 locally forcibly bears against the plate-shaped member 53.

### Embodiment 2

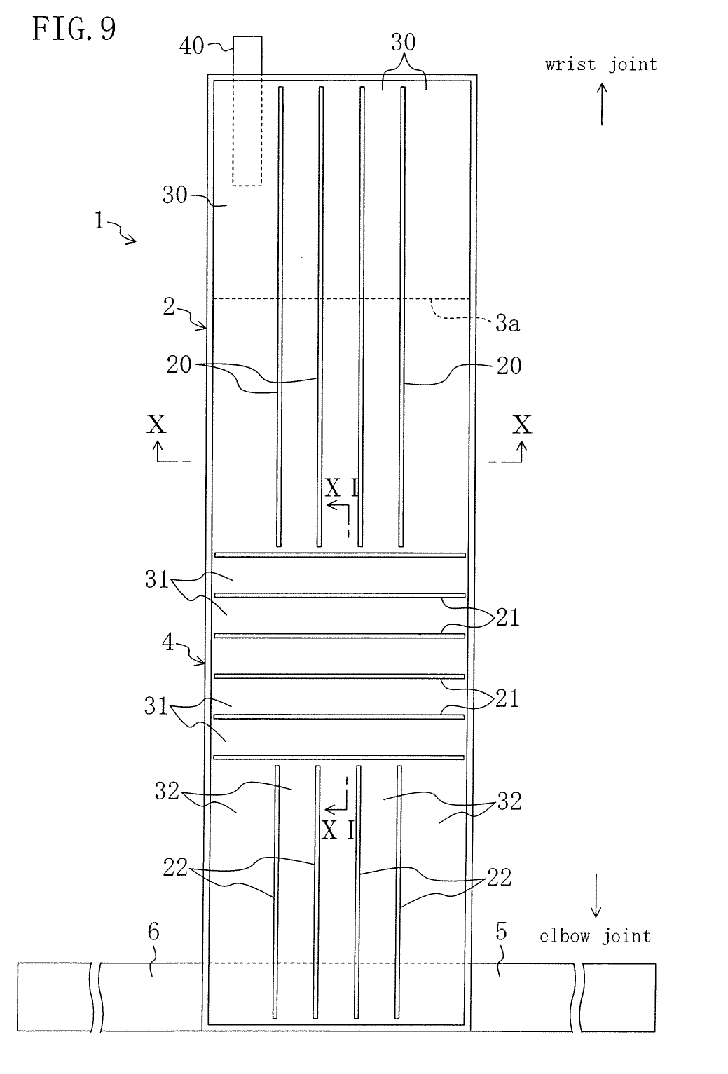

FIG. 9 shows an upper limb restraint device 1 according to Embodiment 2 of the invention. The upper limb restraint device 1 of Embodiment 2 is different from that of Embodiment 1 in their material and the structures of the base 2 and the elbow joint supporting part 4 but otherwise substantially the same. Therefore, a detailed description is given below of different points from Embodiment 1.

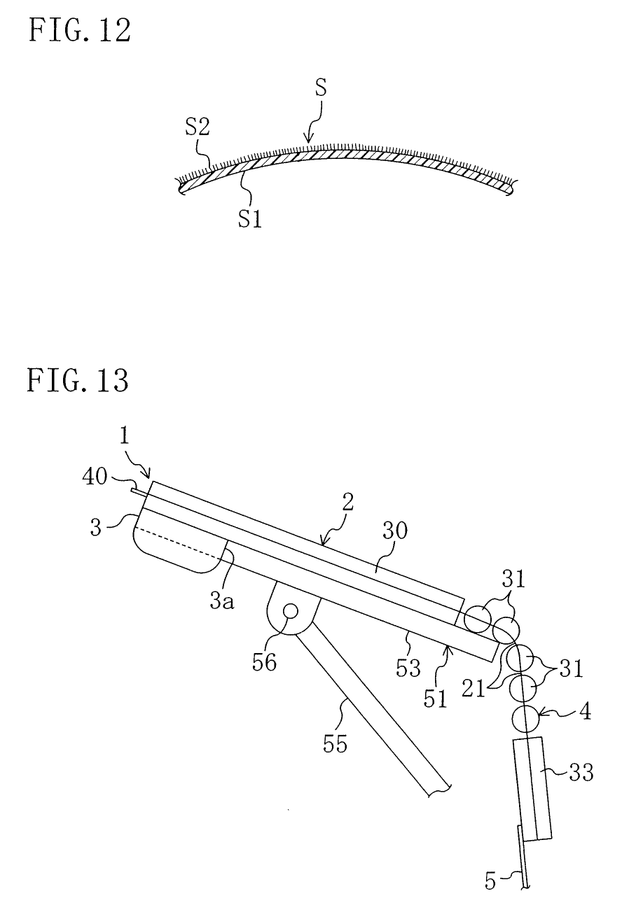

The base 2 and the elbow joint supporting part 4 of the upper limb restraint device 1 is formed, as shown in FIG. 12, using two sheet members S in each of which a nonwoven fabric S2 is adhered to one side of an airtight resin film S1. These sheet members S are formed in a substantially rectangular shape and their peripheral parts are welded in an airtight manner to each other with the nonwoven fabric S2 on the outside.

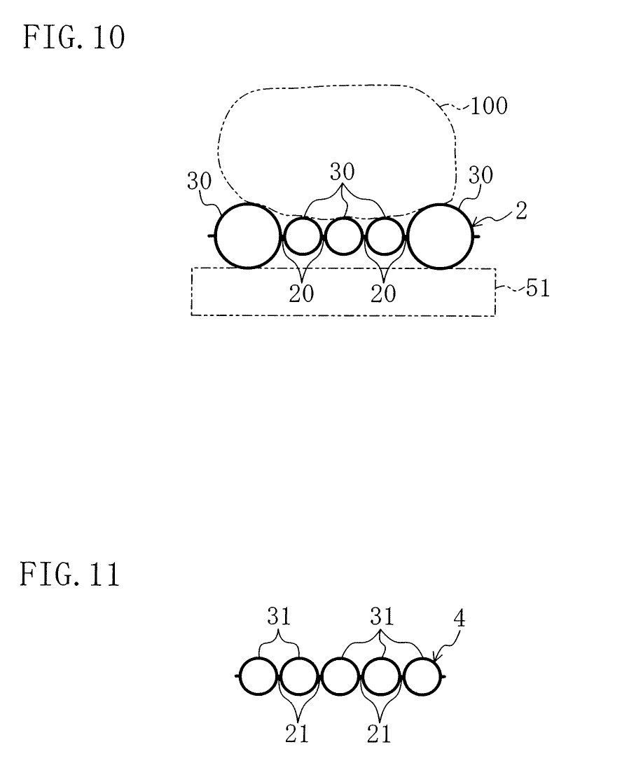

The base 2 has four welded parts 20 provided at spaced intervals in the width direction of the upper limb restraint device 1 to extend in the longitudinal direction thereof. The welded parts 20 are parts where both the sheet members S are welded to each other. As also shown in FIG. 10, by the welded parts 20, five base-side hollow parts 30 of cylindrical shape are defined in the base 2 side by side in the width direction of the base 2. These base-side hollow parts 30 are communicated with each other at both ends in the longitudinal direction of the upper limb restraint device 1. The base-side hollow parts 30 and 30 at both widthwise ends of the base 2 have the same configuration and a larger diameter than the other three base-side hollow parts 30, 30 and 30 in the widthwise middle of the base 2. The three base-side hollow parts 30, 30 and 30 in the widthwise middle of the base 2 have the same configuration.

An inflation valve 40 is provided at an end of the base 2 close to the wrist joint. The inflation valve 40 is communicated with the base-side hollow part 30 located at one widthwise end of the base 2 and has a well-known structure composed of a check valve generally provided such as at an inflation aperture.

The elbow joint supporting part 4 adjoins the base-side hollow parts 30. The elbow joint supporting part 4 has six welded parts 21 provided at spaced intervals in the longitudinal direction of the upper limb restraint device 1 to extend in the width direction thereof. As shown in FIG. 11, by these welded parts 21, five first elbow joint supporting part-side hollow parts 31 of cylindrical shape are defined in the elbow joint supporting part 4 side by side in the longitudinal direction of the upper limb restraint device 1. These first elbow joint supporting part-side hollow parts 31 are communicated with each other at both ends in the width direction of the upper limb restraint device 1. The first elbow joint supporting part-side hollow parts 31 have the same configuration. Furthermore, the first elbow joint supporting part-side hollow parts 31 are communicated with the base-side hollow parts 30.

The portion of the elbow joint supporting part 4 located towards the strings 5 and 6 has four welded parts 22 provided at spaced intervals in the width direction of the upper limb restraint device 1 to extend in the longitudinal direction thereof. By these welded parts 22, second elbow joint supporting part-side hollow parts 32 are defined in the elbow joint supporting part 4 in the same manner as the base-side hollow parts 30. The second elbow joint supporting part-side hollow parts 32 are communicated with the first elbow joint supporting part-side hollow parts 31.

The upper limb rest engagement part 3 is formed by welding a rectangular-shaped sheet material to the sheet member S constituting part of the base 2 and has the shape of a pocket having an opening 3a only at its end close to the elbow joint 102. The material making the first and second strings 5 and 6 is the same as that making the sheet member S. The first and second strings 5 and 6 are welded to the sheet member S. Since the first and second strings 5 and 6 are made of resin, they can be cut with ease if they are too long.

In using the upper limb restraint device 1, air is injected through the inflation valve 40 into the base-side hollow parts 30. The air injected into the base-side hollow parts 30 flows into the first elbow joint supporting part-side hollow parts 31 and the second elbow joint supporting part-side hollow parts 32. Thus, the base-side hollow parts 30, the first elbow joint supporting part-side hollow parts 31 and the second elbow joint supporting part-side hollow parts 32 inflate. Then, as shown in FIG. 13, the upper limb restraint device 1 is set on the upper limb rest 51 in the same way as in Embodiment 1.

Thereafter, the upper limb 100 of the patient A is brought into an arm-raised position and then laid on the top surface of the base 2. In this case, the base-side hollow parts 30 function as a cushion to prevent the upper limb 100 from locally forcibly bearing against the upper limb rest 51. Furthermore, as shown in FIG. 10, since the base-side hollow parts 30 and 30 at both ends of the base 2 in the width direction of the upper limb restraint device 1 have a larger diameter than those in the middle, the upper limb 100 can sink onto the widthwise middle portion of the base 2 and can be thereby supported by the base-side hollow parts 30 and 30 at both ends of the base 2. As a result, the upper limb 100 can be further stabilized on the base 2.

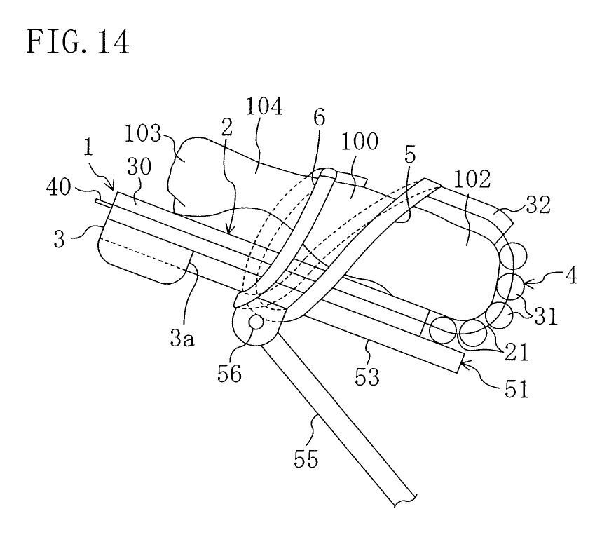

Then, the elbow joint supporting part 4 is folded upward. In this case, since the first elbow joint supporting part-side hollow parts 31 extend in the width direction of the upper limb restraint device 1 and are arranged side by side at longitudinally spaced intervals, they do not interfere with the folding work but provide smooth folding. The elbow joint supporting part 4 covers the elbow joint 102 of the upper limb 100 and, then, as shown in FIG. 14, the first and second strings 5 and 6 are tied like Embodiment 1.

After used, the upper limb restraint device 1 is detached from the upper limb rest 51 and then deflated by opening a hole, for example, in any of the base-side hollow parts 30. Thus, the upper limb restraint device 1 can be folded up compactly and disposed of.

Also according to the upper limb restraint device 1 of Embodiment 2, the same effects as that of Embodiment 1 can be obtained. In addition, since the nonwoven fabric S2 exists on the surface of the upper limb restraint device 1, it can absorb sweat from the patient A during surgery, which gives a good sense of use.

Furthermore, since the upper limb restraint device 1 is provided with the inflation valve 40, the upper limb restraint device 1 can be carried to the point of use in a compact form without injecting air into the hollow parts 30 to 32 before use and then inflated through the inflation valve 40 at the point of use to form the hollow parts 30 to 32. Furthermore, since the hollow parts 30 to 32 contain injected air only, the upper limb restraint device 1 can have a light weight.

In the upper limb restraint device 1 of Embodiment 2, the respective numbers and configurations of hollow parts 30, 31 and 2 can be appropriately selected. The position of the inflation valve 40 may be somewhere in the elbow joint supporting part 4.

Although in Embodiments 1 and 2 two strings 5 and 6 are provided on the elbow joint supporting part 4, the number of strings provided may be one or more than two.

The base 2, the upper limb rest engagement part 3 and the elbow joint supporting part 4 of the upper limb restraint device 1 may be joined together after they are formed as separate elements.

Although in the above embodiments both the upper limbs 100 are restrained on their respective upper limb rests 51 by two upper limb restraint devices 1, only one upper limb 100 may be restrained on the associated upper limb rest 51 by a single upper limb restraint device 1.

The surface of the upper limb restraint device 1 may be coated with antibacterial coating, for example, a 2-methacryloyloxyethyl phosphorylcholine polymer (MPC polymer). Examples of such an MPC polymer include Lipidure-CR1701, Lipidure-CR1702, Lipidure-BG, Lipidure-CM0203, Lipidure-CM5206, Lipidure-CM5206E and Lipidure-CM5208E all made by NOF corporation. These MPC polymers have good biocompatibility and high antibacterial activity and, therefore, are suitable as an antibacterial coating agent for the upper limb restraint device 1. The MPC polymer coating may be applied only to the base 2 of the upper limb restraint device 1 or may be applied only to the elbow joint supporting part 4.

Alternatively, the antibacterial coating applied to the surface of the upper limb restraint device 1 may be, for example, polyhexamethylene biguanide (PHMB) coating.

Still alternatively, the base 2, the upper limb rest engagement part 3, the elbow joint supporting part 4, the first and second strings 5 and 6, the cushion member 10 and the sheet member S constituting part of the upper limb restraint device 1 may be made of resin blended with PHMB.

Furthermore, although not shown, the surface of the upper limb restraint device 1 may be provided with a blood circulation promoter. The blood circulation promoter is composed of two nonwoven fabric sheets laid one on another in the thickness direction and metal powder sandwiched between the nonwoven fabric sheets. The surface of the metal powder is coated with titanium oxide.

Now, a description is given of a method of manufacturing the blood circulation promoter. First, titanium oxide-coated metal powder is obtained. The metal powder can be manufactured, for example, using the apparatuses and methods disclosed in Published Japanese Patent Applications Nos. 2000-61314 and 2004-344687. Specifically, although not shown, a gravity-feed blaster is prepared as the manufacturing equipment. An unprocessed material made of metal powder yet to be coated with titanium oxide is also prepared. The blaster may be anything including siphon type and other types as long as it is of air type.

Then, the unprocessed material is put in a processing chamber of the blaster and titanium powder is sprayed through a spray nozzle onto the unprocessed material by compressed air. The mean particle size of the titanium powder is within the range from 40 to 100 μm. The spraying pressure is selected within the range from 0.4 to 0.6 MPa and the distance between the spray nozzle and the unprocessed material is selected within the range from 100 to 250 mm. The inner diameter of the spray nozzle is selected within the range from 5 to 7 mm.

When titanium powder sprayed through the spray nozzle hits the unprocessed material, heat energy is produced because of changes in speed of the titanium powder between before and after the hit to the unprocessed material surface and thereby causes local temperature increase on the surfaces of the unprocessed material and titanium powder. Thus, the titanium powder is heated on the surface of the unprocessed material and titanium in the powder is activated and adsorbed onto the unprocessed material surface and undergoes an oxidative reaction with oxygen in the atmosphere. As a result, titanium oxide-coated metal powder is obtained. In the course of formation of the titanium oxide coating, the titanium powder gradually increases its temperature, so that titanium gradually penetrates the unprocessed material. Therefore, as titanium penetrates from the surface to the inside of the unprocessed material, it becomes more deficient in oxygen, so that the titanium oxide coating has a so-called oxygen deficit tilting structure. In other words, the titanium oxide coating has a chemical structure of TiO₂ in the surface of the unprocessed material but has a chemical structure of TiO in the inside of the unprocessed material. The metal powder thus obtained is placed between the two nonwoven fabric sheets and the nonwoven fabric sheets are then bonded to each other to confine the metal powder therebetween. The mesh size of the nonwoven fabric is selected at such a narrow value that metal powder thereinside cannot drop out.

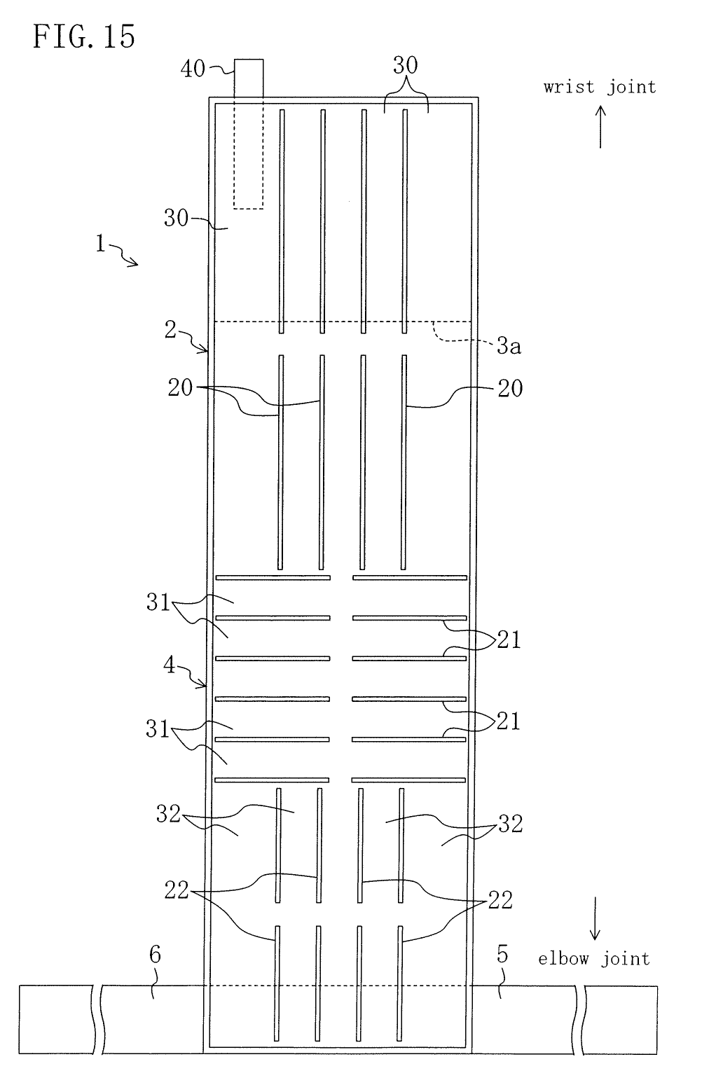

Furthermore, as in Modification 1 of Embodiment 2 shown in FIG. 15, the welded parts 20, 21 and 22 of the upper limb restraint device 1 may be intermittently provided in the longitudinal direction of the upper limb restraint device 1. Thus, air injected through the inflation valve 40 can be allowed to smoothly flow into the hollow parts 30, 31 and 32.

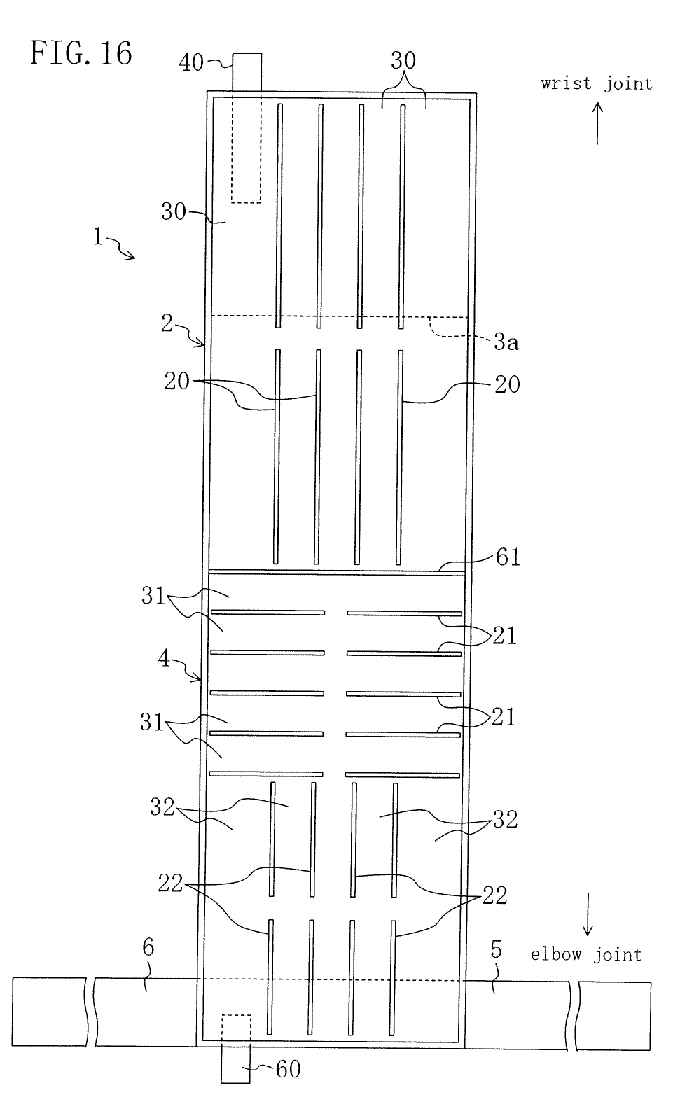

Furthermore, as in Modification 2 of Embodiment 2 shown in FIG. 16, another inflation valve 60 may be provided in addition to the inflation valve 40. The inflation valve 60 is disposed at an end of the elbow joint supporting part 4 close to the strings 5 and 6. When two inflation valves 40 and 60 are provided in this manner, a partition 61 is disposed between the region of the base-side hollow parts 30 and the region of the first elbow joint supporting part-side hollow parts 31 to separate the former from the latter. The partition 61 is formed, like the welded parts 20, by welding the sheet members S to each other. Therefore, air injected through the inflation valve 40 flows only into the base-side hollow parts 30 while air injected through the inflation valve 60 flows into the first elbow joint supporting part-side hollow parts 31 and the second elbow joint supporting part-side hollow parts 32. Thus, the internal pressure of the base-side hollow parts 30 can be made different from the internal pressure of the first elbow joint supporting part-side hollow parts 31 and the second elbow joint supporting part-side hollow parts 32. The number of inflation valves may be three or more.

In injecting air through the inflation valves 40 and 60, the operator or nurse may inspire air orally, inject air with a pump or the like or inject gas directly from a cylinder filled with carbon dioxide or nitrogen.

### INDUSTRIAL APPLICABILITY

As can be seen from the above description, the upper limb restraint device according to the present invention is suitable for restraint of the upper limbs of a patient on the upper limb rests of the surgical table with the upper limbs raised, for example, during surgery on his chest or abdomen.

1. An upper limb restraint device for restraining an upper limb of a patient under surgery on an upper limb rest with the upper limb raised, the upper limb restraint device comprising:
a base placed between the upper limb rest and the upper limb;
an upper limb rest engagement part provided at an end of the base close to the wrist joint of the upper limb and configured to engage with an end of the upper limb rest close to the wrist joint;
an elbow joint supporting part provided at the other end of the base close to the elbow joint of the upper limb and configured to cover the elbow joint of the upper limb laid on the upper limb rest from the underside to the upper side of the elbow joint; and
a string extending from a portion of the elbow joint supporting part covering the upper side of the elbow joint and capable of being bound around the upper limb rest.

2. The upper limb restraint device of claim 1, wherein the upper limb rest engagement part is formed in the shape of a pocket capable of covering a portion of the upper limb rest close to the wrist joint.

3. The upper limb restraint device of claim 1, wherein
the base is made of fabric, and
the elbow joint supporting part is formed by folding the fabric.

4. The upper limb restraint device of claim 1, wherein the string comprises a plurality of said strings extending from separate points on the elbow joint supporting part.

5. The upper limb restraint device of claim 1, further comprising a warming device that warms the upper limb.

6. The upper limb restraint device of claim 5, wherein the warming device is formed by containing a gel warming material capable of warming the upper limb in a bag member and disposed between the upper limb rest and the upper limb.

7. The upper limb restraint device of claim 1, further comprising a cushion member placed between the upper limb rest and the upper limb.

8. The upper limb restraint device of claim 7, wherein the cushion member comprises a bag member into which air is injected.

9. The upper limb restraint device of claim 7, wherein the cushion member is restrained together with the upper limb on the upper limb rest by the string.

10. The upper limb restraint device of claim 1, wherein the base includes a hollow part.

11. The upper limb restraint device of claim 10, wherein the hollow part is provided with an inflation valve through which air is injected into the hollow part.

12. The upper limb restraint device of claim 10, wherein the hollow part comprises at least three said hollow parts arranged side by side in a width direction of the upper limb rest and the hollow parts at both ends of the base in the width direction of the upper limb rest are formed to have a larger size than the hollow part in the middle of the base.
